(19) United States
(12) Patent Application Publication
(10) Pub. No.: US 2018/0015150 A1
(43) Pub. Date: Jan. 18, 2018

(54) THERAPEUTIC INTERVENTION GUIDANCE SYSTEM FOR ATOPIC ECZEMA AND A METHOD OF PROVIDING THERAPEUTIC INTERVENTION GUIDANCE

(71) Applicant: Sphingobrain Co. Ltd., Daejeon (KR)

(72) Inventors: Beyong Deog PARK, Englewood Cliffs, NJ (US); Sekyoo JEONG, Daejeon (KR); Kyong-Oh SHIN, Chungcheongbuk-do (KR); HyunJong KIM, Seoul (KR); Kyungho PARK, Gangwon-do (KR)

(21) Appl. No.: 15/648,440

(22) Filed: Jul. 12, 2017

Related U.S. Application Data
(60) Provisional application No. 62/360,971, filed on Jul. 12, 2016.

(51) Int. Cl. A61K 38/46; A61K 9/00; A61Q 19/00; A61B 5/00; G06F 19/00

(52) U.S. Cl. CPC A61K 38/465; A61Q 19/007; C12Y 301/03016; A61B 5/443; A61K 9/0014; A61B 5/4839; G06F 19/325

(57) ABSTRACT

A therapeutic intervention guidance system for atopic eczema includes a device measuring a skin surface moisture level and a Trans-Epidermal Water Loss (TEWL) value of a subject, a process comparing the skin surface moisture level and the TEWL value with a predetermined level and value, and an outcome providing a therapeutic intervention guidance based on the comparison. A method of providing therapeutic intervention guidance for atopic eczema includes measuring a skin surface moisture level and a Trans-Epidermal Water Loss (TEWL) value of a subject, comparing the skin surface moisture level and the TEWL value with a predetermined level and value, and providing a therapeutic intervention guidance based on the comparison.

This application claims the benefit of U.S. Provisional Application No. 62/360,971, filed on Jul. 12, 2016, which is incorporated by reference for all purposes as if fully set forth herein.

### BACKGROUND OF THE INVENTION

### Field of the Invention

The present invention relates to a therapeutic intervention guidance system for atopic eczema and a method of providing therapeutic intervention guidance using the therapeutic intervention guidance system.

### Discussion of the Related Art

Atopic eczema (AE) or atopic dermatitis (AD), is one of the most common forms of dermatitis. As a chronic, non-contagious inflammatory skin disease, AE is believed to be caused by an immune dysfunction and skin barrier function impairment, triggered by various environmental factors. People who develop atopic dermatitis commonly have a higher incidence of other atopic diseases, such as asthma, conjunctivitis and allergic rhinitis (atopic march or allergic march). While AE may occur in people of any age, it often starts in infants aged 2-6 months. Around 90% of patients with AE experience the onset of disease before the age of 5 years. 75% of people experience marked improvement in the severity of their atopic dermatitis by the age of 14 years. The remaining 25% of sufferers continue to have significant relapses during their adult life In the US, AE affects approximately 10-20% of children and 1% of adults. Worldwide prevalence rates for AE in children range from approximately 3% in Iran and China to 20% in Australasia, the UK and Scandinavia.

There is a need to develop a therapeutic intervention guidance system for preventive treatment or reactive treatment of AE.

### SUMMARY OF THE INVENTION

A therapeutic intervention guidance system for atopic eczema includes a device measuring a skin surface moisture level and a Trans-Epidermal Water Loss (TEWL) value of a subject, a process comparing the skin surface moisture level and the TEWL value with a predetermined level and value, and an outcome providing a therapeutic intervention guidance based on the comparison.

The therapeutic intervention guidance includes usage of a topical calcineurin product.

A method of providing therapeutic intervention guidance for atopic eczema includes measuring a skin surface moisture level and a Trans-Epidermal Water Loss (TEWL) value of a subject, comparing the skin surface moisture level and the TEWL value with a predetermined level and value, and providing a therapeutic intervention guidance based on the comparison.

The therapeutic intervention guidance includes usage of a topical calcineurin product.

The therapeutic intervention guidance further includes stop using the topical calcineurin product.

The therapeutic intervention guidance includes using a skin-care procedure.

The skin-care procedure includes using a moisturizer.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is schematic diagram showing TEWL data with appropriate guidance and without guidance.

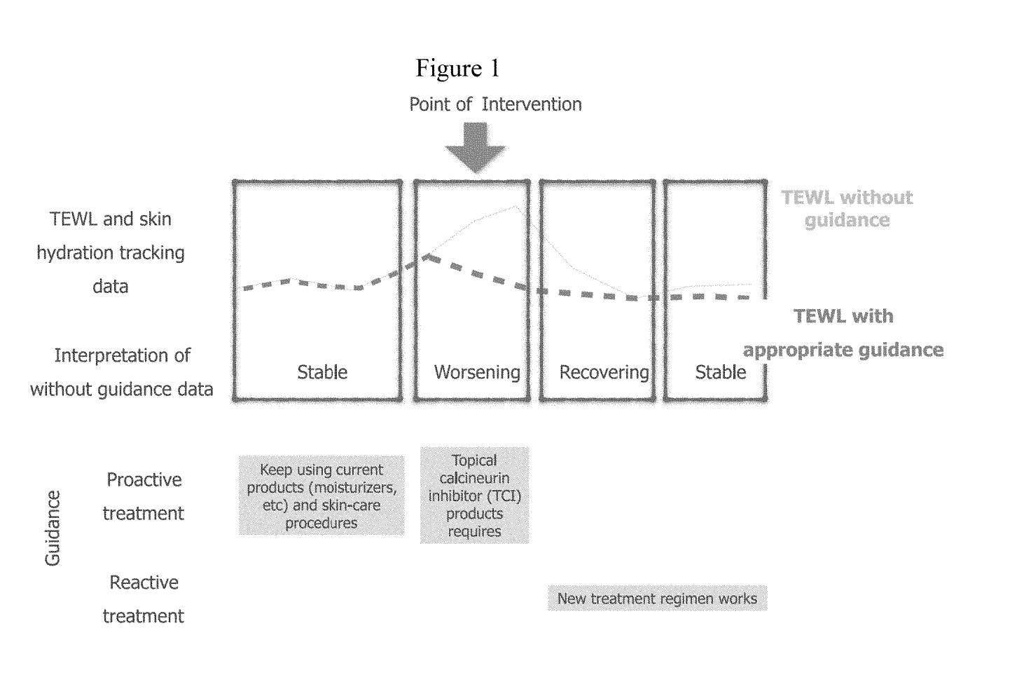

FIG. 2 is schematic diagram showing skin hydration data with appropriate guidance and without guidance.

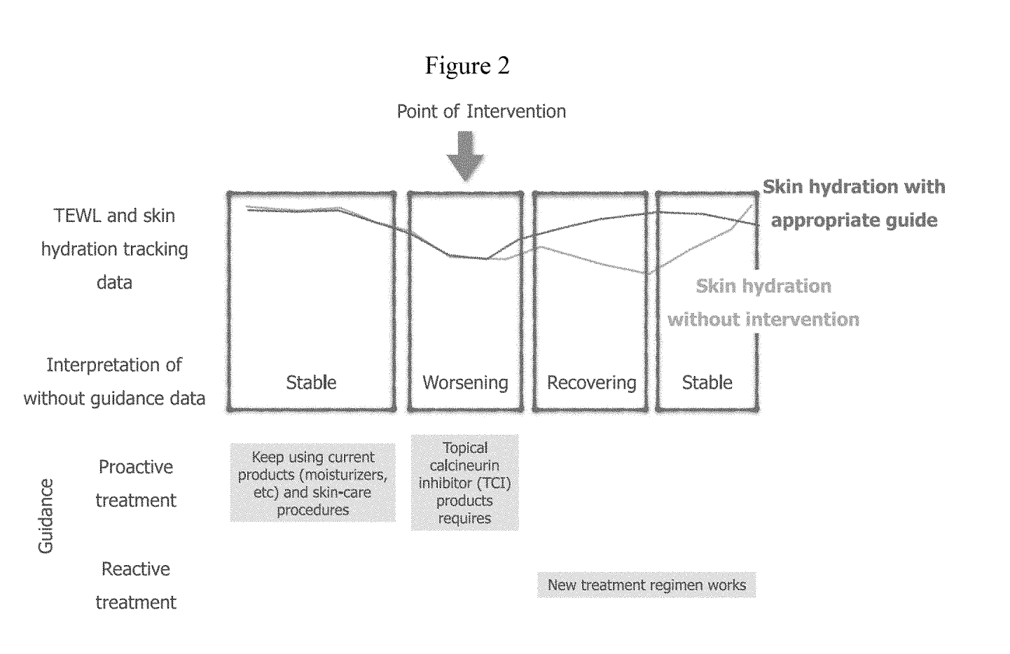

### DETAILED DESCRIPTION OF THE ILLUSTRATED EMBODIMENTS

Reference will now be made in detail to embodiments of the present invention, example of which is illustrated in the accompanying drawings.

Trans-Epidermal Water Loss (TEWL) is defined as the insensible water depletion from inside a body via the epidermal layer, and reflects skin barrier function. Skin hydration is the water content in the skin and on its surface, including water that comes from transpiration. Skin hydration level may be, for example, obtained by measuring the thermal conductivity of the skin of a subject.

A skin function measurement device can measure Trans-epidermal water loss (TEWL) value and skin hydration level at the same time. Thus, the change of skin functions in atopic eczema patients can be tracked. Tracking of skin functions data and using an algorithm, a guidance system for AE management can be established.

As a proactive treatment guidance system, it can provide an alarming signal for the usage of topical calcineurin inhibitor (TCI) products when the data enters pre-determined range (alarming zone). Once the skin function data restore normal range, then patients or caregivers can stop using TCI products. As a reactive treatment guidance system, it can provide an information about the therapeutic efficacy of certain treatment regimens, such as topical pharmaceuticals, systemic pharmaceuticals, behavioral corrections, environment controls, and food intake. Data controlled under normal range imply the current treatment regimens are effective and patients or caregivers can keep those regimens. Caregivers and sufferers can check their skin conditions at any time and any place using the guidance system. According to the skin condition, a therapeutic intervention guidance will be given.

The therapeutic intervention guidance system simultaneously measures skin surface moisture level and Trans-Epidermal Water Loss (TEWL), both of which evaluate the level of skin barrier function. Results are available immediately, and have been proven comparable to those achieved by professional clinical testing equipment. After the measurement, the healing can begin.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A therapeutic intervention guidance system for atopic eczema comprising:
a device measuring a skin surface moisture level and a Trans-Epidermal Water Loss (TEWL) value of a subject,
a process comparing the skin surface moisture level and the TEWL value with a predetermined level and value, and
an outcome providing a therapeutic intervention guidance based on the comparison.

2. The therapeutic intervention guidance system of claim 1, wherein the therapeutic intervention guidance includes usage of a topical calcineurin product.

3. A method of providing therapeutic intervention guidance for atopic eczema comprising:
measuring a skin surface moisture level and a Trans-Epidermal Water Loss (TEWL) value of a subject,
comparing the skin surface moisture level and the TEWL value with a predetermined level and value, and
providing a therapeutic intervention guidance based on the comparison.

4. The method of claim 3, wherein the therapeutic intervention guidance includes usage of a topical calcineurin product.

5. The method of claim 3, wherein the therapeutic intervention guidance further includes stop using the topical calcineurin product.

6. The method of claim 3, wherein the therapeutic intervention guidance includes using a skin-care procedure.

7. The method of claim 6, wherein skin-care procedure includes using a moisturizer.
